(19) United States
(12) Patent Application Publication
(10) Pub. No.: US 2022/0172818 A1
(43) Pub. Date: Jun. 2, 2022

(54) COMPUTER ASSISTED SURGERY SYSTEM AND METHOD TO PROCESS AND COMMUNICATE PLANNING DATA

(71) Applicant: INTELLIJOINT SURGICAL INC., KITCHENER (CA)

(72) Inventors: RICHARD TYLER FANSON, STONEY CREEK (CA); ANDRE NOVOMIR HLADIO, WATERLOO (CA); JUSTIN AARON MICHAEL, VANCOUVER (CA); JOSEPH ARTHUR SCHIPPER, KITCHENER (CA); LUCAS ELI VAN DE MOSSELAER, WATERLOO (CA)

(21) Appl. No.: 17/438,109

(22) PCT Filed: Mar. 5, 2020
(86) PCT No.: PCT/CA2020/050293

§ 371 Date: Sep. 10, 2021

(51) Int. Cl. G16H 20/40; G16H 40/63

(52) U.S. Cl. CPC G16H 20/40; G16H 40/63

(57) ABSTRACT

Provided are systems and methods to facilitate processing and communicating planning data for computer assisted surgery (CAS) procedures. Smart compression may be performed to reduce operational data (e.g. for encoding and/or communicating).

### FIELD

The following relates to computer assisted surgery and to processing and communicating planning data for computer assisted surgery.

### BACKGROUND

An intra-operating room (intra-OR) computer assisted surgery (CAS) system provides a tool to a surgeon to perfume a procedure such as, but not limited to, a hip, knee, shoulder or cranial surgical procedure. During such a procedure, the intra-OR CAS system tracks objects such as surgical tools and parts of a patient's body in a space and provides anatomical measurements as well as relative measurements. The intra-OR CAS system may be provided with instructions to define a workflow and provide a graphical user interface to work through the surgical procedure, displaying measurements and/or relative positions, etc. Such measurements and/or relative positions maybe displayed in association with patient specific images. Often, a surgeon will perform pre-operative or other surgical planning (e.g. using a planning system and software) to define a plan for the procedure where the plan is configured to provide data to the intra-OR CAS system for use during the procedure. A plan may include data defining a 3D model of patient anatomy, which is typically patient specific, target data, anatomic measurements, etc. for use during the procedure.

The planning process may have access to and store patient health information including individually identifiable heath information regarding a patient for whom the procedure is planned.

There is needed a system and method to process and communicate planning data such as to an intra-OR CAS system.

### SUMMARY

Various aspects will be apparent to one of ordinary skill in the art including computing device aspects. Many instances of respective computing devices are shown in an architecture/network for processing, communicating and using planning data.

In one aspect there is provided a computing device comprising: a processor coupled to a storage device, the storage device storing instructions, which when executed by the processor, configure the computing device to provide a planning system to: store planning data for a patient in relation to a surgical procedure, the planning data including or associated with identifying data for the patient; and, communicate the planning data to a computer assisted surgery (CAS) planning data processing system to prepare encoded operational data for delivery to an intra-operating room (intra-OR) CAS system located in an operating room to facilitate performance of the surgical procedure.

The planning data may be communicated without any identifying data for the patient.

The computing device may be further configured to: receive from the CAS planning data processing system at least one of: the encoded operational data and a retrieval data wherein the retrieval data is useful to retrieve at least some operational data from the CAS planning data processing system; and, communicate the at least one of the encoded operational data and the retrieval data for delivery to the intra-OR CAS system. To communicate the at least one of the encoded operational data and the retrieval data may comprise communicating to an intermediate device the at least one of the encoded operational data and the retrieval data in association with identifying data for the patient for confirming the patient in the operating room, the at least one of the encoded operational data and the retrieval data for delivery from the intermediate device without delivering the identifying data to the intra-OR CAS system.

The computing device may be configured to perform data authentication.

To perform data authentication may comprise: communicating a planning data reference with the planning data to the CAS planning data processing system for providing as part of or with the encoded operational data to the intra-OR CAS system; and providing the planning data reference in a separate communication for use to cross-check against the planning data reference received at the intra-OR CAS system.

The at least one of the encoded operational data and the retrieval data may comprise a QR code.

The computing device may remove identifying data for the patient from the planning data prior to communicating the planning data.

The computing device may receive input from a user and define the planning data responsive to the input.

The computing device may be configured to, at least one of: receive imaging data for the patient, the imaging data comprising identifying data for the patient; and use the imaging data to define the planning data; and receive or define a 3D model of patient anatomy; and use the 3D model to define the planning data. The planning data communicated to the CAS planning data processing system may include at least one of the imaging data and the 3D model.

The computing device may: communicate re-identification information in association with the planning data; and receive the re-identification information in association with the at least one of the encoded operational data and the retrieval data to facilitate association of the at least one of the encoded operational data and the retrieval data with the patient.

In a second aspect, there is provided a computing device comprising: a processor coupled to a storage device, the storage device storing instructions, which when executed by the processor, configure the computing device to provide a computer assisted surgery (CAS) planning data processing system to: receive from a planning system planning data for a patient in relation to a surgical procedure; process the planning data to define operational data for an intra-operating room (intra-OR) CAS system located in an operating room to facilitate performance of the surgical procedure; encode the operational data to define encoded operational data for delivery to the intra-OR CAS system; and communicate at least one of the encoded operational data and a retrieval data for delivery to the intra-OR CAS system, the retrieval data useful to retrieve at least some of the operational data from the CAS planning data processing system.

The planning data may be communicated without identifying data for the patient.

The computing device may communicate the encoded operational data for delivery to the intra-OR CAS system via at least one of a) an intermediate mobile device to be present in the OR; b) the planning system; and c) via a communication network without using the intermediate mobile device and the planning system.

To communicate the encoded operational data via the planning system may facilitate the planning system to communicate to the intermediate device the encoded operational data in association with identifying data for the patient for confirming the patient in the operating room, the encoded operational data for delivery from the intermediate device without delivering the identifying data to the intra-OR CAS system.

The encoded operational data may comprise a QR code.

To process the planning data may comprise at least one of: performing a completeness check to ensure expected data for a procedure is received; performing a data confirmation against one or more clinical models to verify a correctness of the planning data; and performing a data conversion from one type or format to another.

The computing device may perform data authentication.

To process the planning data, to encode the operational data or to both process the planning data and encode the operational data may comprise performing smart compression to reduce an amount of operational data.

The computing device may define the encoded operational data from a minimum base of the operational data comprising at least a minimum amount of operational data which permits operations of intra-OR CAS system to facilitate performance of the procedure. The operational data remaining un-encoded in the encoded operational data defines further operational data and wherein the instructions configure the computing device to define the retrieval data to facilitate obtaining the further operational data intra-OR CAS system.

The computing device may: receive re-identification information in association with the planning data; and communicate the re-identification information is association with the at least one of the encoded operational data and retrieval data to facilitate association of the encoded operational data with the patient by the planning system.

In a third aspect there is provided a computing device comprising: a processor coupled to a storage device, the storage device storing instructions, which when executed by the processor, configure the computing device to provide an intermediate device to: receive from a computer assisted surgery (CAS) planning data processing system encoded operational data for delivery to an intra-operating room (intra-OR) CAS system located in an operating room to facilitate performance of a surgical procedure for a patient, the encoded operational data encoding planning data for the surgical procedure; and provide the encoded operational data for delivery to the intra-OR CAS system.

The encoded operational data may encode the planning data without identifying data for the patient.

The computing device may receive the encoded operational data in association with identifying data for the patient for confirming the patient in the operating room, the encoded operational data for delivery from the intermediate device without delivering the identifying data to the intra-OR CAS system.

The encoded operational data may comprise a QR code. The computing device may display the QR code to optically transmit the encoded operational data to the intra-OR CAS system.

The computing device may perform data authentication.

The encoded operational data may comprise at least a minimum amount of operational data which permits operations of the intra-OR CAS system to facilitate performance of the procedure. The operational data remaining un-encoded in the encoded operational data may define further operational data and the encoded operational data may be communicated by the intermediate mobile device in association with or comprising retrieval data to facilitate obtaining the further operational data by intra-OR CAS system.

In a fourth aspect there is provided a computing device comprising: a processor coupled to a storage device defining an intra-operating room (intra-OR) computer assisted surgery (CAS) system for locating in an operating room to facilitate performance of a surgical procedure for a patient, the storage device storing instructions, which when executed by the processor, configure the computing device to: receive, from a CAS planning data processing system, encoded operational data, the encoded operational data encoding planning data for the surgical procedure; and use the encoded operational data to provide the CAS procedure.

The encoded operational data may encode planning data without identifying data for the patient.

To use the encoded operational data may comprise at least one of: decoding; decompressing; and authenticating.

The encoded operational data may comprise a QR code.

To receive may comprise receiving the encoded operational data from an intermediate mobile device present in the OR. The intermediate device may be configured to receive the encoded operational data in association with identifying data for the patient for confirming the patient in the operating room, the encoded operational data received from the intermediate device without receiving the identifying data for the patient.

The computing device may receive the encoded operational data via a camera of the intra-OR CAS system.

The encoded operational data may comprise at least a minimum amount of operational data which permits operations of the intra-OR CAS system to facilitate performance of the procedure. The operational data remaining un-encoded in the encoded operational data may define further operational data stored by the CAS planning data processing system and the encoded operational data may be communicated by the intermediate device in association with or comprising retrieval data to facilitate obtaining the further operational data by intra-OR CAS system.

In a fifth aspect there is provided a computing device comprising: a processor coupled to a storage device, the storage device storing instructions, which when executed by the processor, configure the computing device to provide a computer assisted surgery (CAS) planning data processing system to: process planning data for a patient in relation to a surgical procedure to define operational data for an intra-operating room (intra-OR) CAS system located in an operating room to facilitate performance of the surgical procedure; communicate at least one of the operational data and a retrieval data for delivery to the intra-OR CAS system, the retrieval data useful to retrieve at least some of the operational data from the CAS planning data processing system.

The computing device may be configured to perform planning operations to define the planning data. The computing device may be configured to receive planning data from a planning system configured to define the planning data. The computing device may be configured to receive planning data in other manners, such as by user input (e.g. manual entry), etc.

The computing device may encode the operational data to define encoded operational data for delivery to the intra-OR CAS system.

To encode the operation may comprise encoding as a matrix bar code for optical communication to the intra-OR CAS system.

To process the planning data may comprise performing smart compression of the planning data.

In accordance with any aspect, to perform smart compression may comprise: defining the operational data to comprise a look-up code for use by the intra-OR CAS system to retrieve operational data stored in a library of data of the intra-OR CAS system.

In accordance with any aspect, to perform smart compression may comprise: processing the planning data by computing parameters corresponding to a parameterized anatomical model used by the intra-OR CAS system, wherein the parameterized anatomical model may be a statistical shape model, or any other parameterizable model.

In accordance with any aspect, to perform smart compression may comprise: processing the planning data comprising a 3D model and a target and compressing the 3D model based on the target data, preferably, by performing non-important data removal;

In accordance with any aspect, to perform smart compression may compress the operational data sufficiently to encode the operational data as encoded operational data in a code, preferably a QR code, for decoding by the intra-OR CAS system.

In accordance with any aspect, to perform smart compression may comprise at least one of: performing data generalization comprising transforming a model into a pre-defined coordinate system; lowering a data precision; using a generic model to store a surface to represent the model using a statistical shape model; and performing standard lossless compression.

Method and other aspects will be apparent.

### BRIEF DESCRIPTION OF THE DRAWINGS

FIG. 1 is a block diagram of a computer network for CAS planning and for performing intra-OR CAS in accordance with an example.

FIG. 2 is a block diagram of a computing device in accordance with an example, instances of which may be a configured as respective components of the computer network of FIG. 1.

FIGS. 3A, 3B, 3C and 3D are flowcharts of operations of computing machine components of the computer network of FIG. 1 in accordance with an example.

FIGS. 4A, 4B and 4C are flowcharts of operations of FIGS. 3B and 3D in further detail in accordance with an example.

The present inventive concept is best described through certain embodiments thereof, which are described herein with reference to the accompanying drawings, wherein like reference numerals refer to like features throughout. It is to be understood that the term invention, when used herein, is intended to connote the inventive concept underlying the embodiments described below and not merely the embodiments themselves. It is to be understood further that the general inventive concept is not limited to the illustrative embodiments described below and the following descriptions should be read in such light. More than one inventive concept may be shown and described and each may standalone or be combined with one or more others unless stated otherwise.

### DETAILED DESCRIPTION

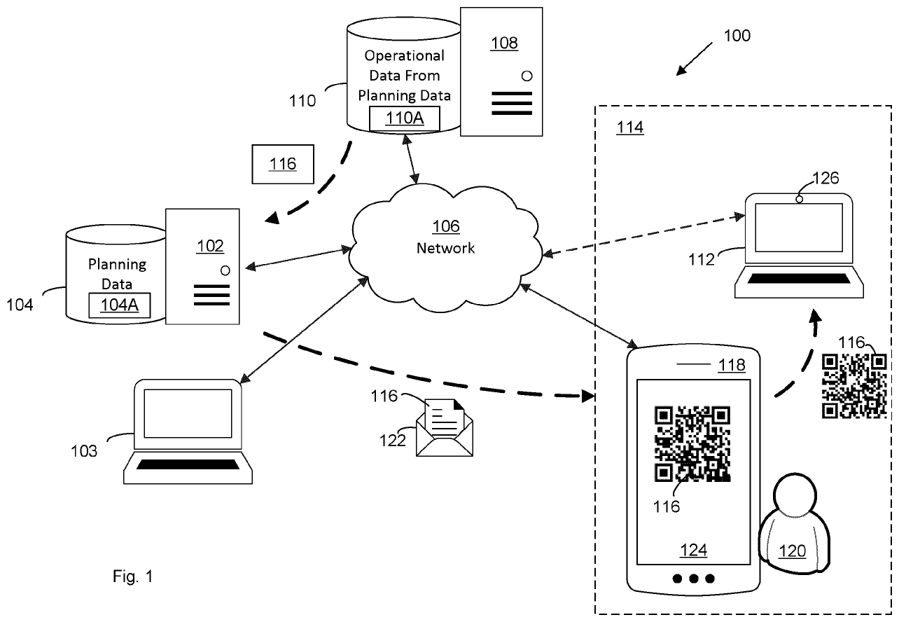

FIG. 1 is a block diagram of a computer network 100 for CAS planning and for performing intra-OR CAS in accordance with an example. There is shown a planning system 102 (typically a server) and planning data store 104. Planning system 102 is configured (e.g. via a planning application (software) to perform planning for a surgical procedure. In the present example, it is defined in accordance with a client/server model to communicate with a planning client device 103. Another model could be used.

Planning system 102 may receive patient imaging data (not shown) in accordance with one or more modalities such computerized tomography (CT), magnetic resonance imaging (MRI), x-ray, or a proprietary format (such as from EOS imaging, Paris, France). Planning operations may also receive patient identifying information, such as patent name, address, employer name, address, health care provider information, payor information, etc. Planning operations may produce planning data 104A for example, including clinical targets, a 3D model, anatomic measurements, etc., related to the surgery to achieve a desired outcome. By way of example, for hip surgery (clinical targets may comprise cup inclination/anteversion, 3D model of a pelvis, anatomic measurements may comprise ASIS points, Pubis, Hip Center, Pelvic Tilt, etc. At least some of this planning data 104A may be used by an intra-OR CAS system to provide computer assisted surgery such as by tracking objects, making and presenting measurements using workflow and a graphical user interface to assist the surgeon to achieve the (planned) targets.

Planning system 102 and its data store 104 may be owned and/or operated by a first entity (not shown) having a business relationship with hospital management (not shown) of a hospital (not shown) in which the computer assisted surgery is to be performed. The surgeon (not shown), a user of planning client device 103, may also have a relationship with such hospital management and often with the first entity as well. These relationships need not be direct and may be indirect, e.g. via one or more intermediaries. The respective relationships include obligations relating to the storage, use and communication of health information, particularly individually identifying health information.

Planning data 104A for a procedure may be communicated, such as via a network 106, to a CAS planning data processing system 108 having a data store 110. CAS planning data processing system 108 is configured to process planning data 104A to produce operational data 110A for use by an intra-OR CAS system 112 during the procedure. Intra-OR CAS system 112 is located in an operating room (OR) 114 of the hospital and CAS planning data processing system 108 is usually remotely located relative to the OR 114 and thus is remote from intra-OR CAS system 112. CAS planning data processing system 108 may receive and process planning data 104A for use by a plurality of intra-OR CAS systems located at different operating rooms which may be in different hospitals. Though the term hospital is used, other environments are contemplated.

The CAS planning data processing system 108 and intra-OR CAS system 112 may be owned and/or operated by a second entity, independent of the first entity. The second entity may not wish to receive health information which identifies or could be used to identify an individual. The first entity and/or hospital may desire not to communicate health information which identifies or could be used to identify an individual to the second entity.

Planning data 104A received from planning system 108 may comprise de-identified health information such that identifiers of the individual patient (or of relatives, employers, and household members of the patient) are not present such that there is no actual knowledge that the remaining information could be used to identify the patient.

In one example, planning data may comprise a single file following the DICOM standard. Where the planning data comprises a 3D model, the 3D model may in an STL file format. The 3D model may be encapsulated in the DICOM file using the Encapsulated STL Information Object Definition (IOD) from the DICOM standard. Binary STL data may be included in the “Encapsulated Document” DICOM attribute (tag 0042,0011). Additional planning data information may be included in one or more other DICOM attributes in the Encapsulated STL IOD with Value Representations that allow long strings of data, such at Long Text (LT), Unlimited Characters (UC), or Unlimited Text (UT). “Patient Comments” is one such attribute.

It will be understood that planning data may be received in other manners by CAS planning data processing system 108. Planning data may be received such as via user input (e.g. manual entry), etc. For example, if planning is done using non-digital media (e.g. acetate templates), or even with digital media but no connection exists between CAS planning data processing system 108 and planning system 102, data can be entered manually, for example, using a browser or other interface that provides a form for data entry.

CAS planning data processing system 108 may provide to planning system 102 operational data 110A for a specific patient as encoded operational data 116 for providing to intra-OR CAS system 112. Operational data 110A may be encoded as encoded operational data 116 in the form of one or more Quick Response (QR) codes or other matrix barcode or similar encoding such as for optical transmission to intra-OR CAS system 112 as further described. Planning system 102 may receive encoded operational data 116 and store same with its planning data 104A (e.g. in data store 104 and in association with individually identifiable information for the patient). CAS planning data processing system 108 may provide an interface to a planning system user such as a surgeon/user of device 103. CAS planning data processing system 108 may require approval of the operational data 110A before encoding and communicating same for delivery to intra-OR CAS system 112 via planning system 102 or otherwise. Operations of CAS planning data processing system 108 may permit a surgeon (user) to update planning data/operational data, recall previously communicated encoded operational data and invoke a communicating of updated encoded operational data for delivery to intra-OR CAS system 112.

As the surgical procedure draws near in time, for example, the encoded operational data 116 may be communicated to a representative of the first entity (e.g. to a mobile device 118 of user 120) who is present in the OR 114 during the procedure. Communication may be in other means such as by printed document, via a native application, etc. Communication may be via planning system 102 or from CAS planning data processing system 108 without communication through planning system 102, per se. It is understood that should the communication be an email from CAS planning data processing system 108 to mobile device 118, such an email may be directed through an (enterprise) email server owned or operated by or on behalf of the first entity.

The encoded operational data 116 may be a portion of a communication such as an email 122 (SMS, native application message, etc.) The communication may include (or link to) individually identifiable information for the patient which may be used to confirm against the patient (patient data from the hospital) present in the OR during the procedure.

The encoded operational data 116 may be presented such as via a display device 124 of mobile device 118 or in a printed document for optical transmission to intra-OR CAS system 112 via a camera 126 of (or coupled to) intra-OR CAS system 112. Intra-OR CAS system 112 may decode and use the encoded operational data 116 to facilitate performance of the CAS procedure.

In some examples, intra-OR CAS system 112 has a connection to network 106 and in some examples it does not have a connection. Thus the connection is represented by a dashed connection line representing a connection that may not be present. In some examples, intra-OR CAS system 112 does not have a connection because it is spotty or temporarily unavailable. Encoded operational data 116 may encode at least a minimum base of operational data 110A to perform the procedure using intra-OR CAS system 112 without having intra-OR CAS system 112 communicate with CAS planning data processing system 108.

Optionally or in addition, the encoded operational data 116 may provide or be communicated with retrieval data (as described further) to facilitate requesting further operational data via CAS planning data processing system 108. Intra-OR CAS system 112 may communicate via network 106 for such further operational data. As will be described, in some examples, only a retrieval data is communicated to intra-OR CAS system 112 (without encoded operational data 116 per se) to retrieve the operational data 110A from CAS planning data processing system 108.

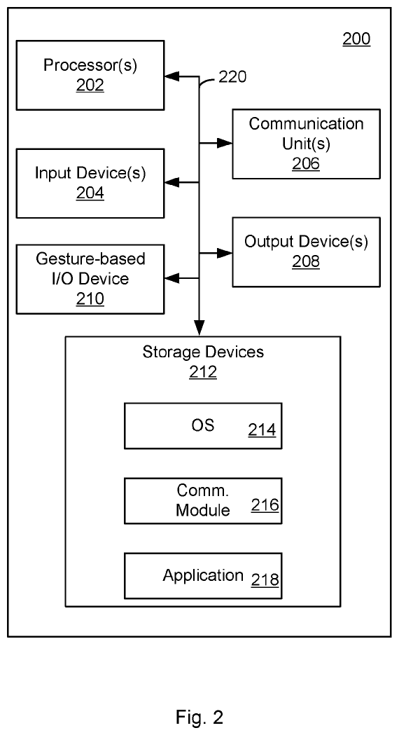

FIG. 2 is a block diagram of a computing device 200. Computing device 200 comprises one or more processors 202 (e.g. one or more CPUs, GPUs, ASICs, FPGAs, etc.), input devices 204, which may include a camera, one or more communication units 206 (e.g. for wired or wireless communication), one or more output devices 208 (e.g. a speaker, light, etc.), and a gestures-based I/O device such a touch screen 210 to receive input and present output visually. Computing device 200 also comprises one or more storage devices 212 (e.g. memory, disks, etc.) to store data including an operating system (OS) 214, communication module 216 to facilitate wired and/or wireless communication and one or more applications (e.g. Application 218), such as for any of various computer tasks and functions. Instances of computing device 200 may be configured with different form factors (e.g. mobile device (laptop, tablet, smartphone, wearable, etc.), desktop or PC, server, etc.) and have more or fewer components. For example a server may not have a gesture based I/O device. Some instances may have other built in or coupled display devices that need not be gesture based. Other input devices may include a keyboard, buttons, pointing device, microphone, etc.

An instance of computing device 200 may be configured as a planning system 102. Application 218 may comprise a planning application (e.g. server side). Other applications may include a data store (e.g. a database application), communications such as an email server and/or email client etc. An instance of computing device 200 may be configured as a planning client device 103. Application 218 may comprise a planning application (e.g. client side). Other applications may include communication applications, etc.

An instance of computing device 200 may be configured as a CAS planning data processing system 108. Application 218 may comprise an application to process planning data 104A, compress and encode operational data 110A and communicate encoded operational data 116. It is understood that more than one application may be provided for such tasks. Other applications may include a data store (e.g. a database application).

An instance of computing device 200 may be configured as an intra-OR CAS system 112. Application 216 may include a CAS application such as described. Application 218 or another application may be configured to receive encoded operational data 116 using camera 124 as described and to communicate with server 108 for further operational data. Intra-OR CAS system 112 may be coupled to components of a localization system (not shown) to track objects in space. Such components may include an optical sensor (e.g. a camera) to receive signals from markers respectively coupled to the objects to enable a determination of relative position of the objects in space. Other sensors providing input may include positional sensors such as accelerometers, gyroscopes, magnetometers, etc. The others sensors may be in a housing with the optical sensor.

An instance of computing device 200 may be configured as mobile device 118. Application 118 may comprise an email client application to communicate email. Other applications and configurations may provide features/functions to display a QR code. A native client application may be configured to communicate with planning system 102 to receive planning data 104A, QR code, etc.

While only a single planning system 102 and a single planning client device 103 are shown in computer network 100, it will be understood that any single planning system 102 may communicate with each planning client device 103 of a plurality of respective planning client devices such as for multiple users. It will also be understood that multiple instances of respective planning systems 102 may communicate with CAS planning data processing system 108. Furthermore, an individual instance of a planning client device 103 may be configured to communicate with two or more respective planning systems 102, for example, to work with different providers of implant prosthetics. A respective planning client device 103 may be configured via respective planning client applications, for example, to enable planning with each respective planning system 102.

In FIG. 1, dashed arrows show a flow of communications to deliver the encoded operational data 116 from CAS planning data processing system 108 to planning system 102 then to mobile device 118 (an intermediate device or intermediate mobile device) and then, optically communicated, to intra-OR CAS system 112 in OR 114. It is understood that mobile device 118 need not be present in the OR 114 to receive the communication from planning system 102.

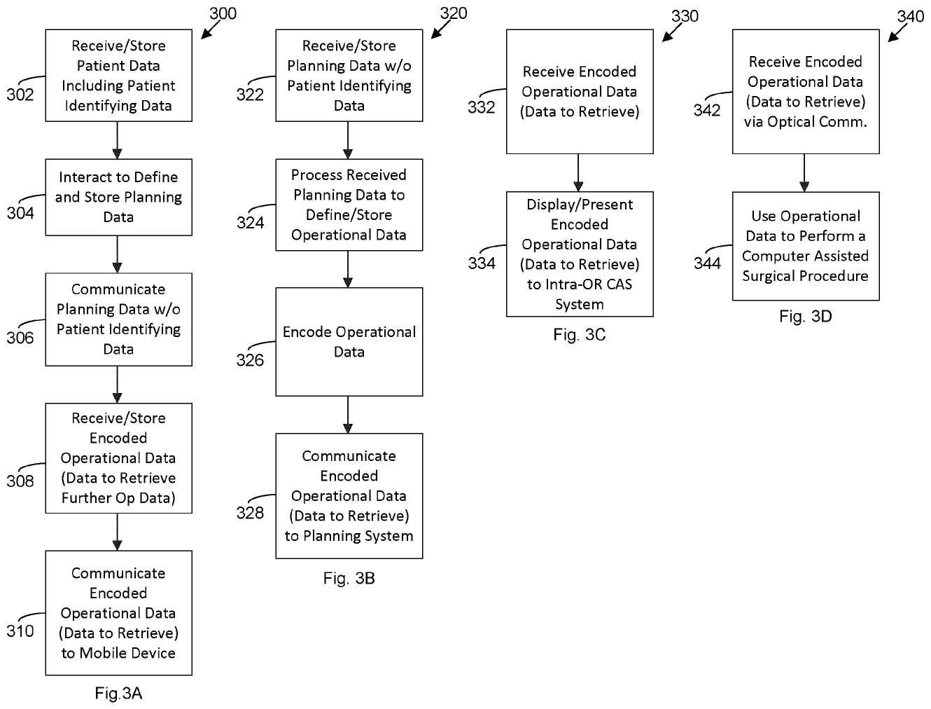

FIGS. 3A-3D are flowcharts of respective operations 300, 320, 330 and 340 of the computing device components 102, 108, 118 and 112 respectively of the computer network of FIG. 1 in accordance with an example. Regarding FIG. 3A and operations 300, at step 302, planning system 102 receives and stores patient data including patient identifying data. Patient data may include images of patient anatomy from one or more modalities. At step 304, planning system 102 (e.g. via its applications) interacts with client side application of planning client device 103 to plan the procedure, defining and storing planning data 104A. Interaction may comprise receiving user input and defining the planning data responsive to the user input. As noted, planning data 104A may comprise clinical targets, imaging data, 3D model of patient anatomy, anatomic measurements, visualizations of implants, implant make/model, simulations of ranges of motion, etc. Clinical targets in hip surgery may comprise: Cup inclination/anteversion; Femoral implant version; Hip center location; Desired leg length/offset. In other surgeries, targets may comprise: Glenoid implant angles (shoulder); and Pedicle screw trajectory (spine). Anatomic measurements in hip surgery may comprise: Native hip inclination/anteversion; Native femoral version; Initial leg length/offset; and Saggital pelvic mobility. These and others will be apparent to those of ordinary skill in the art.

At 306, planning data 104A without patient identifying data is communicated to CAS planning data processing system 108. Planning system 102 (e.g. via an application) may be configured to remove data including any applicable metadata from records, images, etc. sent to CAS planning data processing system 108 such that any identifying data is de-identified. Planning system 102 is configured to only transmit personal health data that is non-identifying so that the data sent is compliant with applicable privacy legislation and rules. For example, in relation to the United States, the planning data 104A communicated is de-identified health information where there is no restriction on the use or disclosure of same under the Health Insurance Portability and Accountability Act of 1996 (“HIPAA”), Pub. L. 104-191 and the rules and regulations thereunder. The planning data 104A may include or be transmitted with a unique identifying number, characteristic or code for re-identification (herein after “re-identification information”).

At 308, at least one of encoded operational data 116 and retrieval data to obtain at least some of the operational data is received such as in the form of a QR code. The encoded operational data 116 may include or be associated with retrieval data for CAS system 112 to use to retrieve further operational data from CAS planning data processing system 108 that is not encoded in the encoded operational data or even to receive all operational data. The AR code may encode the operational data and the retrieval data may be separate therefrom. The QR code may only encode (store) the retrieval data and no encoded operational data may be received.

The at least one of encoded operational data 116 and retrieval data is stored such as in planning data store 110. Any re-identification information provided is returned with the communication of the at least one of encoded operational data 116 and retrieval data so as to facilitate the storing of same in association with the planning data 104A and/or patient identifying data by planning system 102. It will be appreciated that, in the present example, encoded operational data 116 encodes planning data for a surgical procedure for the patient without any identifying information for the patient.

At 310, at least one of encoded operational data 116 and retrieval data is communicated to mobile device 118. The communication may be an email (e.g. 122) or other form. The communication typically includes patient identifying data for use to confirm a patient during a CAS procedure in the OR but without providing the patient identifying data to CAS system 112. Mobile device 118 acts as an intermediate (mobile) device for delivery of the at least one of encoded operational data 116 and retrieval data to intra-OR CAS system 112. Intra-OR CAS system 112 may be configured with workflow to prompt input confirming that the patient is identified, for example, to continue the procedure. Any retrieval data if received separately from encoded operational data may also be communicated to the mobile device for delivery to intra-OR CAS system 112.

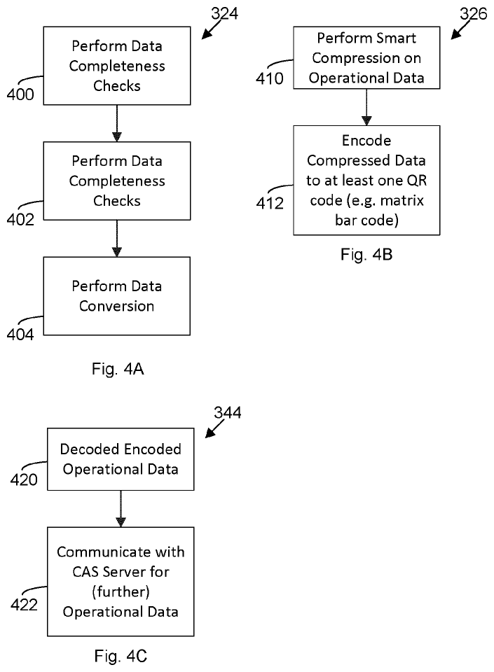

Regarding FIG. 3B and operations 320 of CAS planning data processing system 108, at step 322, planning data 104A without patient identifying data is received. The communication includes re-identification information. At 324 planning data 104A is processed to define and store operational data 110. Further detail is described with reference to FIG. 4A. At 326 operations encode at least a minimum base (a minimum subset) of the operational data to produce encoded operational data 116. The minimum base is at least a minimum amount of operational data which permits operations of intra-OR CAS system 112 to facilitate performance of the CAS procedure. The remainder of the operational data not coded defines further operational data. Further detail is described with reference to FIG. 4B.

Retrieval data (e.g. a unique identifying number, characteristic or code) may be generated and communicated with the encoded operational data 116 to facilitate obtaining the further operational data from CAS planning data processing system 108 by intra-OR CAS system 112. Retrieval data may comprise a network address/URL, etc. using Web techniques, etc. To communicate the retrieval data with the encoded operational data may comprise encoding the retrieval data as a part of the encoded operational data or including it as a separate data item. It is also understood that the retrieval data need not be communicated, per se, as it may be derivable from the encoded operational data. For example, if the operational data is unique over the set of patients, a hash of the encoded data (if not the encoded data itself) could be used as retrieval data by the intra-OR CAS system 112, providing it to the CAS planning data processing system 108 to request further data. In another example, the retrieval data may only be sent and not any encoded operational data. The retrieval data may be useful to retrieve any and all of the operational data. The retrieval data (without any operational data) also may be encoded in a QR code.

Communication of the at least one of the encoded operational data 116 and retrieval data from CAS planning data processing system 108 and communication (e.g. of planning data) to CAS planning data processing system 108 may be performed in accordance with any required and/or desired security measures. Encryption may be utilized.

At 328 operations communicate the at least one of the encoded operational data 116 and retrieval data to planning system 102 for further communication. The retrieval data is communicated optionally for reasons mentioned—such data may not be necessary to communicate because as all operational data is encoded, the retrieval data is encoded as part of the operational data item, or the retrieval data is derivable from the operational data/encoded operational data. The communication is associated with any re-identification information received so that planning system 102 may appropriately store the at least one of the encoded operational data 116 and retrieval data.

Regarding FIG. 3C and operations 330 of mobile device 118, at step 332 the encoded operational data 116 is received such as in a communication comprising an email. Communications to mobile device 118 from planning system 102 (and vice versa) may be performed in accordance with any required and/or desired security measures. Encryption may be used. The communication may be from CAS planning data processing system 108 and also may be performed in accordance with any required and/or desired security measures.

When received from planning system 102, the email typically includes patient identifying information to confirm patient identity and ensure that the at least one of the encoded operational data 116 and retrieval data is for the correct patient. The mobile device is operated by a member (e.g. an employee or contractor) of the first entity. Such an entity is often further associated with the conduct of the surgery as a supplier of prosthetic implants, and/or surgical tools for the procedure. A member of the entity is typically present in the OR. The member may receive the patient identifying data and the at least one of the encoded operational data and retrieval data to the member's mobile device. The member (with any hospital staff, etc.) may confirm the patient identity. The mobile device may be operated to display the QR code to intra-OR CAS system 112 (e.g. camera 126) to optically transfer the at least one of the encoded operational data 116 and retrieval data. In another example, the QR code (encoded operational data 116) is communicated via a printed (e.g. paper) document from planning system 102 and/or user 120. In this manner, neither CAS planning data processing system 108 nor intra-OR CAS system 112 receives patient identifying data.

Regarding FIG. 3D and operations 340 of intra-OR CAS system 112, at step 342 the at least one of the encoded operational data 116 and the retrieval data is received. Its correctness may be verified such as via a checksum, hash, unique identifier, etc. The checksum, hash, unique identifier, etc. may be encoded in the data communicated to the intra-OR CAS system. The checksum, hash, unique identifier, etc. may be communicated such as to mobile device 118 with a planning report (e.g. in a separate or same communication with the AR code). This data may be cross-checked to verify. At 344 at least some of the operational data 110A is used to facilitate performance of a computer assisted surgical procedure. Further detail is provided with reference to FIG. 4C.

As noted, intra-OR CAS system 112 may be configured with workflow to prompt input confirming that the patient is identified, for example, to enable use of the operational data. Confirmation of the correctness of the patient and his/her data may be performed by comparison of patient identifying data communicated to mobile device 118 (but not to intra-OR CAS system 112) and patient identifying data on charts, tags and other records in the OR, including information coupled to the patient.

Further, operational data received and decoded in intra-OR CAS system 112 may be compared to a copy of planning data or operational data also received at mobile device 118.

FIGS. 4A-4C are flowcharts of operations of FIGS. 3B and 3D in further detail in accordance with an example. FIG. 4A provides examples of operations 324 to process the planning data. Processing the planning data may comprise any one or more of: performing various completeness checks (at 400) to ensure expected data for a procedure is received in the planning data (or at least a minimum of same is present), performing data correctness confirmations (at 402) (e.g. against one or more clinical models, though this is likely to be performed during planning) to confirm targets, etc. are reasonable), and perform data conversions (at 404) (e.g. from one data format or type to a format or type used by intra-OR CAS system 112), etc. This may include transferring data between/among coordinate systems. Processing the data may vary depending on the planning system and its configuration, which may be related to the planning entity/3rd party planning vendor (source of the planning system). The order shown may be different. Data conversion may be performed earlier, for example.

Regarding FIG. 4B and operations 326, at step 410 CAS planning data processing system 108 performs smart compression on the planning data and/or operational data. Smart compression may include, but is not limited to, one or more of the following techniques:
non-important data removal (e.g. removing data representing portions of the model which are not relevant to a plan execution such as to remove a non-operative side of a portion of anatomy or data representing anatomic feature(s) which are not visible or accessible, e.g. the internal facing side of the pelvis);
data generalization (e.g. transform the model into a pre-defined coordinate system, center around hip center);
lowering the data precision (e.g. scale the data to be in the range of −2048 to 2047, transforming each floating point (4 byte) number in to a 12 bit integer (1.5 bytes), and storing a higher precision scaling factor);
using generic models to store the surface (e.g. store the articulating acetabular surface as a sphere and a boundary, or represent the model using statistical shape models);
performing standard lossless compression (e.g. zipping);
storing a library of data on the intra-OR computer, and providing a look-up code in the (encoded) operational data to find the right data. For example, planning data may include a 3D model of the patient anatomy. The intra-OR computer may be configured to perform a registration between intra-operatively collected navigation data and a 3D model (e.g. using a surface matching algorithm, such as the Iterative Closest Point (ICP) algorithm). The 3D model used for registration may be selected from a library of static or parameterizable (parameter adaptable) models available in memory on the intra-OR computer. An anatomical atlas may implemented on the intra-OR computer such that the 3D model used for registration is derived therefrom. The CAS planning data processing system 108 may process the planning data to generate a look-up code and/or parameters that are used as operational data to enable the intra-OR CAS system 112 to retrieve and/or generate a 3D model used for registration.
alternatively, making available a parameterized anatomical model at intra-OR CAS system 112, and providing parameters via the operational data 116. In this case, CAS planning data processing system 108 may process the planning data by computing parameters corresponding to the parameterized anatomical model, and provide these parameters as part of the operational data. The parameterized anatomical model may be a statistical shape model, or any other parameterizable model.
processing the planning data comprising a 3D model and a target, compressing the 3D model based on the target data, for example, using any of the techniques above. For example, the smart compression method may identify regions of the 3D model (planning data) that have a known spatial relationship with the clinical target (planning data). The identified regions may be preserved in their full fidelity, whereas other regions in the 3D model may be compressed (e.g. by limiting precision, parameterization, becoming sparser, or omitting the data).

Via smart compression, planning data may be compressed to the point where operational data may be fully represented by encoded operational data (e.g. a QR code may provide all of the required operational data, including data to generate a 3D model for registration). This is advantageous, since network connectivity of the intra-OR CAS system 112 would not be required to retrieve the operational data, thus alleviating cybersecurity risks.

It will be appreciated that smart compression processing may be performed on planning data, operational data or both. That is, processing planning data to define operational data may utilize the smart compression techniques. Bright line distinctions may not be relevant. Hence, CAS planning data processing system 108 may process the planning data, encode the operational data or both process the planning data and encode the operational data by performing smart compression to reduce an amount of operational data to be encoded.

At 412, operations encode the operational data for communication such as into at least one QR code. The data encoded into the QR code may include a hash or checksum, etc. as authentication data to verify correctness of the operational data and/or planning data when decoded. Such operations by each respective computing device (generator and receiver) may comprise performing data authentication. The hash or checksum may relative to one or both of the planning data and the operational data. That is, there may be a planning data hash to authenticate planning data, an operational data hash to authenticate operational data and/or a combined hash of both planning data and operational data.

As noted above, operations encode at least a minimum base (a minimum subset) of the operational data to produce encoded operational data 116. The minimum base is at least a minimum amount of operational data which permits operations of intra-OR CAS system 112 to facilitate performance of the procedure. The remainder of the operational data not coded defines further operational data. For example, a minimum base of operational data to be useful intra-operatively in a hip related procedure may include: surgical targets (e.g. cup inclination, anteversion, desired leg length numbers). In this example, the intra-OR CAS system would be able to proceed with surgical targets confirmed to be associated with the patient undergoing the surgery. The amount of data required to encode the information of this example is low enough to facilitate encoding on a QR code. Retrieval data (e.g. a unique identifying number, characteristic or code) may be generated and communicated with the encoded operational data 116 to facilitate obtaining the further operational data from the CAS system 108 by intra-OR CAS system 112. The retrieval data may be encoded as a part of the encoded operational data 116 or included as a separate data item.

In another example, as described but not shown, the operational data is not encoded into the QR code but the retrieval data is so encoded.

In the present example, the encoded operational data 116, any re-identification information, and any retrieval data (included in the encoded operational data 116 or as a separate data item) are communicated to planning system 102 for delivery to intra-OR CAS system 112. Delivery may be through an intermediate (mobile) device such as mobile device 118.

Regarding FIG. 4C and operations 344, at 420 intra-OR CAS system 112 decodes encoded operational data 116. The data may be authenticated by performing data authentication. The decoded data is available for use by workflow, GUI etc. within the intra-OR CAS system 112 to facilitate performance of the CAS procedure. If applicable (e.g. further operational data is stored at data store 110 and intra-OR CAS system 112 has a data connection) and a retrieval code is received or derivable, at 422, intra-OR CAS system 112 may communicate to CAS planning data processing system 108 to receive the further operational data.

In another example, as described but not shown, the operational data is not encoded into the QR code but the retrieval data is so encoded and as such the retrieval data is used to retrieve the operational data from CAS planning data processing system 108.

### Further Examples

While the above examples describe minimizing the transfer of patient identifying information to avoid CAS planning data processing system 108 and intra-OR CAS system 112 receiving same, it is understood that the computing network components could be configured to communicate patient identifying information to CAS planning data processing system 108 for its use and storage. CAS planning data processing system 108 may process patient identifying information as operational data such that patient identifying information becomes encoded in the encoded operational data. Patient identifying information could be provided through to intra-OR CAS system 112 via planning system 102 and mobile device 118.

Email correspondence providing the QR code (e.g. whether encoding operational data or retrieval data or both) may originate from CAS planning data processing system 108.

A planning data reference (e.g. a number) may be generated and included as part of the planning data from planning system 102. The reference number is then included as (encoded) operational data. In the operating room, the 3rd party (e.g. user 120) has access to a “planning report” (such as via mobile device 118 or otherwise from planning system 102), including the reference number. The planning data reference from the planning report and as received and decoded from the operational data may be cross checked to authenticate the operational data (e.g. verify the correct operational data is received at by intra-OR CAS system 112). Rather than a reference number per se, a hash may be used of the planning data. Planning system 102 may generate and provide a hash such as to mobile device 118 with or associated with the planning report. CAS planning data processing system 108 may receive the hash and verify it with the planning data. The hash may be encoded in the operational data and communicated ultimately to intra-OR CAS system 112. The two hashes may be verified such as by hospital staff. For example, the hashes may be presented on respective display screens, and/or print outs, etc. for verification. Such verifications etc. may be employed whether the encoded operational data and/or retrieval data is provided (e.g. as an email) from planning system 102 or CAS planning data processing system 108 to mobile device 118. Encoding or otherwise producing and processing reference numbers or hash data for verification are examples of performing data authentication and respective steps may be performed by any of the computing devices herein. It is understood that data authentication may be configured to authenticate planning data, operational data or both.

It will be understood that utilizing QR codes may enable communication to intra-OR CAS system 112 (e.g. from mobile device 118) without using a communication network, for example, where devices are connected to one another and signals are transmitted between the devices. However, encoded operational data or retrieval data may be communicated to intra-OR CAS system 112 using a communication network. For example, but not limited thereto, the network may be a local network. The local network may be established between intra-OR CAS system 112 and mobile device 118.

The local network may be any of a Bluetooth® network, ZigBee® network, near field communication (NFC) network, Wi-Fi hotspot, or other short range wireless network. Bluetooth is a trademark of the Bluetooth SIG. ZigBee is a trademark of ZigBee Alliance. Sometimes short range networks are referenced as personal area networks (PANs), wireless ad hoc networks, near-me area networks (NANs), etc.

An alternative to a wireless network is use of an audio signal and interface. That is, data may be transmitted (communicated) via an audio jack (audio port) using a form of modulation/demodulation and supporting audio cables to a receiving device configured to receive same.

It will also be understood that CAS planning data processing system 108 may provide data such as encoded operational data and/or retrieval data to intra-OR CAS system 112 via a communication network, for example, without need for an intermediate (mobile) device such as mobile device 118.

In one manner, CAS planning data processing system 108 may provide a secure file hosting service or communicate the data to an external secure file hosting service and the intra-OR CAS system 112 provided with applicable information to utilize the file hosting service to retrieve (e.g. download) the data. For example, the applicable information to utilize the file hosting service may be manually input into intra-OR CAS system 112. Of course, such applicable information may be retrieval data encoded in and received via QR code(s), or other manner, such as from mobile device 118, a print out, etc. to permit the intra-OR CAS system 112 to utilize the file hosting service to obtain the encoded operational data. In one manner CAS planning data processing system 108 may be enabled as a cloud service and intra-OR CAS system 112 enabled to communicate therewith to obtain the data. Other manners will be apparent to those of ordinary skill.

It is understood that aspects from the further examples may be combined with one another and with aspects of the earlier example(s) when logical to do so, even if same may be redundant.

In addition to computing device aspects, a person of ordinary skill will understand that computer program product aspects are disclosed, where instructions are stored in a non-transient storage device (e.g. a memory, CD-ROM, DVD-ROM, disc, etc.) to configure a computing device to perform any of the method aspects described and/or illustrated herein.

Practical implementation may include any or all of the features described herein. These and other aspects, features and various combinations may be expressed as methods, apparatus, systems, means for performing functions, program products, and in other ways, combining the features described herein. A number of embodiments have been described. Nevertheless, it will be understood that various modifications can be made without departing from the spirit and scope of the processes and techniques described herein. In addition, other steps can be provided, or steps can be eliminated, from the described process, and other components can be added to, or removed from, the described systems. Accordingly, other embodiments are within the scope of the following claims.

Throughout the description and claims of this specification, the word “comprise” and “contain” and variations of them mean “including but not limited to” and they are not intended to (and do not) exclude other components, integers or steps. Throughout this specification, the singular encompasses the plural unless the context requires otherwise. In particular, where the indefinite article is used, the specification is to be understood as contemplating plurality as well as singularity, unless the context requires otherwise.

Features, integers characteristics, compounds, chemical moieties or groups described in conjunction with a particular aspect, embodiment or example of the invention are to be understood to be applicable to any other aspect, embodiment or example unless incompatible therewith. All of the features disclosed herein (including any accompanying claims, abstract and drawings), and/or all of the steps of any method or process so disclosed, may be combined in any combination, except combinations where at least some of such features and/or steps are mutually exclusive. The invention is not restricted to the details of any foregoing examples or embodiments. The invention extends to any novel one, or any novel combination, of the features disclosed in this specification (including any accompanying claims, abstract and drawings) or to any novel one, or any novel combination, of the steps of any method or process disclosed.

What we claim is:

1. A computing device comprising:
a processor coupled to a storage device, the storage device storing instructions, which when executed by the processor, configure the computing device to provide a computer assisted surgery (CAS) planning data processing system to:
receive from a planning system planning data for a patient in relation to a surgical procedure;
process the planning data to define operational data for an intra-operating room (intra-OR) CAS system located in an operating room to facilitate performance of the surgical procedure;
encode the operational data to define encoded operational data for delivery to the intra-OR CAS system; and
communicate at least one of the encoded operational data and a retrieval data for delivery to the intra-OR CAS system, the retrieval data useful to retrieve at least some of the operational data from the CAS planning data processing system.

2. The computing device of claim 1 wherein the planning data is communicated without identifying data for the patient.

3. The computing device of claim 1 or claim 2 wherein the instructions configure the computing device to communicate the encoded operational data for delivery to the intra-OR CAS system via at least one of
a) an intermediate mobile device to be present in the OR;
b) the planning system; and
c) via a communication network without communicating through the intermediate mobile device and the planning system.

4. The computing device of claim 3 wherein to communicate the encoded operational data via the planning system facilitates said planning system to communicate to the intermediate mobile device the encoded operational data in association with identifying data for the patient for confirming the patient in the operating room, the encoded operational data for delivery from the intermediate mobile device without delivering the identifying data to the intra-OR CAS system.

5. The computing device of any one of claims 1 to 4 wherein the encoded operational data comprises a QR code.

6. The computing device of any one of claims 1 to 5 wherein to process the planning data comprises at least one of:
performing a completeness check to ensure expected data for a procedure is received;
performing a data confirmation against one or more clinical models to verify a correctness of the planning data; and
performing a data conversion from one type or format to another.

7. The computing device of any one of claims 1 to 6 comprising performing data authentication.

8. The computing device of any one of claims 1 to 7 wherein to process the planning data, encode the operational data or both process the planning data and encode the operational data comprises performing smart compression to reduce an amount of operational data.

9. The computing device of any one of claims 1 to 8 wherein the instructions configure the computing device to define the encoded operational data from a minimum base of the operational data comprising at least a minimum amount of operational data which permits operations of intra-OR CAS system to facilitate performance of the procedure.

10. The computing device of claim 9 wherein the operational data remaining un-encoded in the encoded operational data defines further operational data and wherein the instructions configure the computing device to define the retrieval data to facilitate obtaining the further operational data intra-OR CAS system.

11. The computing device of any one of claims 1 to 10 wherein the instructions configure the computing device to:
receive re-identification information in association with the planning data; and
communicate the re-identification information is association with the at least one of the encoded operational data and retrieval data to facilitate association of the encoded operational data with the patient by the planning system.

12. A computing device comprising:
a processor coupled to a storage device, the storage device storing instructions, which when executed by the processor, configure the computing device to provide a planning system to:
store planning data for a patient in relation to a surgical procedure, the planning data including or associated with identifying data for the patient; and,
communicate the planning data to a computer assisted surgery (CAS) planning data processing system to prepare encoded operational data for delivery to an intra-operating room (intra-OR) CAS system located in an operating room to facilitate performance of the surgical procedure.

13. The computing device of claim 12 wherein the planning data is communicated without any identifying data for the patient.

14. The computing device of claim 12 or claim 13 further configured to:
receive from the CAS planning data processing system at least one of: the encoded operational data and a retrieval data wherein the retrieval data is useful to retrieve at least some operational data from the CAS planning data processing system; and,
communicate the at least one of the encoded operational data and the retrieval data for delivery to the intra-OR CAS system.

15. The computing device of claim 14 wherein to communicate the at least one of the encoded operational data and the retrieval data comprises communicating to an intermediate device the at least one of the encoded operational data and the retrieval data in association with identifying data for the patient for confirming the patient in the operating room, the at least one of the encoded operational data and the retrieval data for delivery from the intermediate device without delivering the identifying data to the intra-OR CAS system.

16. The computing device of any one of claims 12 to 15 wherein the instructions configure the computing device to perform data authentication.

17. The computing device of claim 16 to perform data authentication comprises:
communicating a planning data reference with the planning data to the CAS planning data processing system for providing as part of or with the encoded operational data to the intra-OR CAS system; and
providing the planning data reference in a separate communication for use to cross-check against the planning data reference received at the intra-OR CAS system.

18. The computing device of any one of claims 12 to 17 wherein the at least one of the encoded operational data and the retrieval data comprises a QR code.

19. The computing device of any one of claims 12 to 18 wherein the instructions configure the computing device to remove identifying data for the patient from the planning data prior to communicating the planning data.

20. The computing device of any one of claims 12 to 19 wherein the instructions configure the computing device to receive input from a user and define the planning data responsive to the input.

21. The computing device of claim 20 wherein the instructions configure the computing device to at least one of:
receive imaging data for the patient, the imaging data comprising identifying data for the patient; and use the imaging data to define the planning data; and
receive or define a 3D model of patient anatomy; and use the 3D model to define the planning data.

22. The computing device of claim 21 wherein the planning data communicated to the CAS planning data processing system includes at least one of the imaging data and the 3D model.

23. The computing device of any one of claims 12 to 22 wherein the instructions configure the computing device to:
communicate re-identification information in association with the planning data; and
receive the re-identification information in association with the at least one of the encoded operational data and the retrieval data to facilitate association of the at least one of the encoded operational data and the retrieval data with the patient.

24. A computing device comprising:
a processor coupled to a storage device, the storage device storing instructions, which when executed by the processor, configure the computing device to provide an intermediate device to:
receive from a computer assisted surgery (CAS) planning data processing system encoded operational data for delivery to an intra-operating room (intra-OR) CAS system located in an operating room to facilitate performance of a surgical procedure for a patient, the encoded operational data encoding planning data for the surgical procedure; and
providing the encoded operational data for delivery to the intra-OR CAS system.

25. The computing device of claim 24 wherein the encoded operational data encodes planning data without identifying data for the patient.

26. The computing device of claim 24 or claim 25 wherein instructions configure the computing device to receive the encoded operational data in association with identifying data for the patient for confirming the patient in the operating room, the encoded operational data for delivery from the intermediate device without delivering the identifying data to the intra-OR CAS system.

27. The computing device of any one of claims 24 to 26 wherein the encoded operational data comprises a QR code.

28. The computing device of claim 27 wherein the instructions configure the computing device to display the QR code to optically transmit the encoded operational data to the intra-OR CAS system.

29. The computing device of any one of claims 24 to 28 wherein the instructions configure the computing device to perform data authentication.

30. The computing device of any one of claims 24 to 29 wherein the encoded operational data comprises at least a minimum amount of operational data which permits operations of the intra-OR CAS system to facilitate performance of the procedure.

31. The computing device of claim 30 wherein the operational data remaining un-encoded in the encoded operational data defines further operational data and wherein the encoded operational data is communicated by the intermediate device in association with or comprising retrieval data to facilitate obtaining the further operational data by intra-OR CAS system.

32. A computing device comprising:
a processor coupled to a storage device defining an intra-operating room (intra-OR) computer assisted surgery (CAS) system for locating in an operating room to facilitate performance of a surgical procedure for a patient, the storage device storing instructions, which when executed by the processor, configure the computing device to:
receive, from a CAS planning data processing system, encoded operational data, the encoded operational data encoding planning data for the surgical procedure; and
use the encoded operational data to provide the CAS procedure.

33. The computing device of claim 32 wherein the encoded operational data encodes planning data without identifying data for the patient.

34. The computing device of claim 32 or claim 33 wherein to use the encoded operational data comprises at least one of:
decoding;
decompressing; and
authenticating.

35. The computing device of any one of claims 32 and 34 wherein the encoded operational data comprises a QR code.

36. The computing device of any one of claims 32 to 35 wherein to receive comprises receiving the encoded operational data from an intermediate device present in the OR.

37. The computing device of claim 36 wherein the intermediate device is configured to receive the encoded operational data in association with identifying data for the patient for confirming the patient in the operating room, the encoded operational data received from the intermediate device without receiving the identifying data for the patient.

38. The computing device of any one of claims 32 to 37 wherein the instructions configure the computing device to receive the encoded operational data via a camera of the intra-OR CAS system.

39. The computing device of any one of claims 32 to 38 wherein the encoded operational data comprises at least a minimum amount of operational data which permits operations of the intra-OR CAS system to facilitate performance of the procedure.

40. The computing device of claim 39 wherein the operational data remaining un-encoded in the encoded operational data defines further operational data stored by the CAS planning data processing system and wherein the encoded operational data is communicated by the intermediate device in association with or comprising retrieval data to facilitate obtaining the further operational data by intra-OR CAS system.

41. A computing device comprising:
a processor coupled to a storage device, the storage device storing instructions, which when executed by the processor, configure the computing device to provide a computer assisted surgery (CAS) planning data processing system to:
process planning data for a patient in relation to a surgical procedure to define operational data for an intra-operating room (intra-OR) CAS system located in an operating room to facilitate performance of the surgical procedure;
communicate at least one of the operational data and a retrieval data for delivery to the intra-OR CAS system, the retrieval data useful to retrieve at least some of the operational data from the CAS planning data processing system.

42. The computing device of claim 41 wherein instructions configure the computing device to encode the operational data to define encoded operational data for delivery to the intra-OR CAS system.

43. The computing device of claim 42 wherein to encode the operation comprises encoding as a matrix bar code for optical communication to the intra-OR CAS system.

44. The computing device of any one of claims 41 to 43 wherein to process the planning data comprises performing smart compression of the planning data.

45. The computing device of any one of claims 8, and 9 to 11 as they depend from claims 8 and 44 wherein to perform smart compression comprises:
defining the operational data to comprise a look-up code for use by the intra-OR CAS system to retrieve operational data stored in a library of data of the intra-OR CAS system.

46. The computing device of any one of claims 8, and 9 to 11 as they depend from claims 8 and 44 to 45 wherein to perform smart compression comprises:
processing the planning data by computing parameters corresponding to a parameterized anatomical model used by the intra-OR CAS system, wherein the parameterized anatomical model may be a statistical shape model, or any other parameterizable model.

47. The computing device of any one of claims 8, and 9 to 11 as they depend from claims 8 and 44 to 46 wherein to perform smart compression comprises:
processing the planning data comprising a 3D model and a target and compressing the 3D model based on the target data, preferably, by performing non-important data removal;

48. The computing device of any one of claims 8, and 9 to 11 as they depend from claims 8 and 44 to 47 wherein to perform smart compression compresses the operational data sufficiently to encode the operational data as encoded operational data in a code, preferably a QR code, for decoding by the intra-OR CAS system.

49. The computing device of any one of claims 8, and 9 to 11 as they depend from claims 8 and 44 to 48 wherein to perform smart compression comprises at least one of:
performing data generalization comprising transforming a model into a pre-defined coordinate system;
lowering a data precision;
using a generic model to store a surface to represent the model using a statistical shape model; and
performing standard lossless compression.

50. The computing device of any one of claims 41 to 49 configured to perform planning operations to define the planning data.

51. The computing device of any one of claims 41 to 50 configured to receive planning data from a planning system configured to define the planning data.

52. A method comprising:
storing, by a planning system, planning data for a patient in relation to a surgical procedure, the planning data including or associated with identifying data for the patient; and,
communicating by the planning system the planning data to a computer assisted surgery (CAS) planning data processing system to prepare encoded operational data for delivery to an intra-operating room (intra-OR) CAS system located in an operating room to facilitate performance of the surgical procedure.

53. The method of claim 52 wherein the planning data is communicated without any identifying data for the patient.

54. The method of claim 52 or claim 53 further comprising:
receiving from the CAS planning data processing system at least one of: the encoded operational data and a retrieval data wherein the retrieval data is useful to retrieve at least some operational data from the CAS planning data processing system; and,
communicating the at least one of the encoded operational data and the retrieval data for delivery to the intra-OR CAS system.

55. The method of claim 54 wherein to communicate the at least one of the encoded operational data and the retrieval data comprises communicating to an intermediate device the at least one of the encoded operational data and the retrieval data in association with identifying data for the patient for confirming the patient in the operating room, the at least one of the encoded operational data and the retrieval data for delivery from the intermediate device without delivering the identifying data to the intra-OR CAS system.

56. The method of any one of claims 52 to 55 comprising performing data authentication.

57. The method of claim 56 wherein performing data authentication comprises:
communicating a planning data reference with the planning data to the CAS planning data processing system for providing as part of or with the encoded operational data to the intra-OR CAS system; and
providing the planning data reference in a separate communication for use to cross-check against the planning data reference received at the intra-OR CAS system.

58. The method of any one of claims 52 to 57 wherein the at least one of the encoded operational data and the retrieval data comprises a QR code.

59. The method of any one of claims 52 to 58 comprising removing identifying data for the patient from the planning data prior to communicating the planning data.

60. The method of any one of claims 52 to 59 comprising receiving input from a user and defining the planning data responsive to the input.

61. The method of claim 60 comprising at least one of:
receiving imaging data for the patient, the imaging data comprising identifying data for the patient; and using the imaging data to define the planning data; and
receiving or defining a 3D model of patient anatomy; and using the 3D model to define the planning data.

62. The method of claim 61 wherein the planning data communicated to the CAS planning data processing system includes at least one of the imaging data and the 3D model.

63. The method of any one of claims 52 to 62 comprising:
communicating re-identification information in association with the planning data; and
receiving the re-identification information in association with the at least one of the encoded operational data and the retrieval data to facilitate association of the at least one of the encoded operational data and the retrieval data with the patient.

64. A method comprising:
receiving, at a computer assisted surgery (CAS) planning data processing system and from a planning system, planning data for a patient in relation to a surgical procedure;
processing by the computer assisted surgery (CAS) planning data processing system the planning data to define operational data for an intra-operating room (intra-OR) CAS system located in an operating room to facilitate performance of the surgical procedure;
encoding the operational data to define encoded operational data for delivery to the intra-OR CAS system; and
communicating at least one of the encoded operational data and a retrieval data for delivery to the intra-OR CAS system, the retrieval data useful to retrieve at least some of the operational data from the CAS planning data processing system.

65. The method of claim 64 wherein the planning data is communicated without identifying data for the patient.

66. The method of claim 64 or claim 65 comprising communicating the encoded operational data for delivery to the intra-OR CAS system via at least one of a) an intermediate device to be present in the OR; and b) the planning system.

67. The method of claim 66 wherein to communicate the encoded operational data via the planning system facilitates said planning system to communicate to the intermediate device the encoded operational data in association with identifying data for the patient for confirming the patient in the operating room, the encoded operational data for delivery from the intermediate device without delivering the identifying data to the intra-OR CAS system.

68. The method of any one of claims 64 to 67 wherein the encoded operational data comprises a QR code.

69. The method of any one of claims 64 to 68 wherein to process the planning data comprises at least one of:
performing a completeness check to ensure expected data for a procedure is received;
performing a data confirmation against one or more clinical models to verify a correctness of the planning data; and
performing a data conversion from one type or format to another.

70. The method of any one of claims 64 to 69 comprising performing data authentication.

71. The method of any one of claims 64 to 70 wherein to process the planning data, encode the operational data or both process the planning data and encode the operational data comprises performing smart compression to reduce an amount of operational data.

72. The method of any one of claims 64 to 71 comprising defining the encoded operational data from a minimum base of the operational data comprising at least a minimum amount of operational data which permits operations of intra-OR CAS system to facilitate performance of the procedure.

73. The method of claim 72 wherein the operational data remaining un-encoded in the encoded operational data defines further operational data and wherein the method defines the retrieval data to facilitate obtaining the further operational data intra-OR CAS system.

74. The method of any one of claims 64 to 73 comprising:
receiving re-identification information in association with the planning data; and
communicating the re-identification information is association with the at least one of the encoded operational data and retrieval data to facilitate association of the encoded operational data with the patient by the planning system.

75. A method comprising:
receiving at an intermediate device from a computer assisted surgery (CAS) planning data processing system encoded operational data for delivery to an intra-operating room (intra-OR) CAS system located in an operating room to facilitate performance of a surgical procedure for a patient, the encoded operational data encoding planning data for the surgical procedure; and
providing by the intermediate device the encoded operational data for delivery to the intra-OR CAS system.

76. The method of claim 75 wherein the encoded operational data encodes planning data without identifying data for the patient.

77. The method of claim 75 or claim 76 comprising receiving the encoded operational data in association with identifying data for the patient for confirming the patient in the operating room, the encoded operational data for delivery from the intermediate device without delivering the identifying data to the intra-OR CAS system.

78. The method of any one of claims 75 to 77 wherein the encoded operational data comprises a QR code.

79. The method of claim 78 wherein the instructions configure the computing device to display the QR code to optically transmit the encoded operational data to the intra-OR CAS system.

80. The method of any one of claims 75 to 79 comprising performing data authentication.

81. The method of any one of claims 75 to 80 wherein the encoded operational data comprises at least a minimum amount of operational data which permits operations of the intra-OR CAS system to facilitate performance of the procedure.

82. The method of claim 81 wherein the operational data remaining un-encoded in the encoded operational data defines further operational data and wherein the method comprises communicating the encoded operational data in association with or comprising retrieval data to facilitate obtaining the further operational data by intra-OR CAS system.

83. A method comprising:
receiving, at an intra-operating room (intra-OR) computer assisted surgery (CAS) system located in an OR to facilitate a CAS procedure on a patient and from a CAS planning data processing system, encoded operational data, the encoded operational data encoding planning data for a surgical procedure; and
using the encoded operational data to provide the CAS procedure.

84. The method of claim 83 wherein the encoded operational data encodes planning data without identifying data for the patient.

85. The method of claim 83 or claim 84 wherein to use the encoded operational data comprises at least one of:
decoding;
decompressing; and
authenticating.

86. The method of any one of claims 83 and 85 wherein the encoded operational data comprises a QR code.

87. The method of any one of claims 83 to 86 wherein to receive comprises receiving the encoded operational data from an intermediate device present in the OR.

88. The method of claim 87 wherein the intermediate device is configured to receive the encoded operational data in association with identifying data for the patient for confirming the patient in the operating room, the encoded operational data received from the intermediate device without receiving the identifying data for the patient.

89. The method of any one of claims 83 to 88 comprising receiving the encoded operational data via a camera of the intra-OR CAS system.

90. The method of any one of claims 83 to 89 wherein the encoded operational data comprises at least a minimum amount of operational data which permits operations of the intra-OR CAS system to facilitate performance of the procedure.

91. The method of claim 90 wherein the operational data remaining un-encoded in the encoded operational data defines further operational data stored by the CAS planning data processing system and wherein the encoded operational data is communicated by the intermediate device in association with or comprising retrieval data to facilitate obtaining the further operational data by intra-OR CAS system.

92. A method comprising:
processing, at a computer assisted surgery (CAS) planning data processing system, planning data for a patient in relation to a surgical procedure to define operational data for an intra-operating room (intra-OR) CAS system located in an operating room to facilitate performance of the surgical procedure; and
communicating by the CAS planning data processing system at least one of the operational data and a retrieval data for delivery to the intra-OR CAS system, the retrieval data useful to retrieve at least some of the operational data from the CAS planning data processing system.

93. The method of claim 92 wherein instructions configure the computing device to encode the operational data to define encoded operational data for delivery to the intra-OR CAS system.

94. The method of claim 93 wherein to encode the operation comprises encoding as a matrix bar code for optical communication to the intra-OR CAS system.

95. The method of any one of claims 92 to 94 wherein to process the planning data comprises performing smart compression of the planning data.

96. The method of any one of claims 71, and 72 to 74 as they depend from claims 71 and 95 wherein to perform smart compression comprises:
defining the operational data to comprise a look-up code for use by the intra-OR CAS system to retrieve operational data stored in a library of data of the intra-OR CAS system.

97. The method of any one of claims 71, and 72 to 74 as they depend from claims 71 and 95 to 96 wherein to perform smart compression comprises:
processing the planning data by computing parameters corresponding to a parameterized anatomical model used by the intra-OR CAS system, wherein the parameterized anatomical model may be a statistical shape model, or any other parameterizable model.

98. The method of any one of claims 71, and 72 to 74 as they depend from claims 71 and 95 to 97 wherein to perform smart compression comprises:
processing the planning data comprising a 3D model and a target and compressing the 3D model based on the target data, preferably, by performing non-important data removal;

99. The method of any one of claims 71, and 72 to 74 as they depend from claims 71 and 95 to 98 wherein to perform smart compression compresses the operational data sufficiently to encode the operational data as encoded operational data in a code, preferably a QR code, for decoding by the intra-OR CAS system.

100. The method of any one of claims 71, and 72 to 74 as they depend from claims 71 and 95 to 99 wherein to perform smart compression comprises at least one of:
performing data generalization comprising transforming a model into a pre-defined coordinate system;
lowering a data precision;
using a generic model to store a surface to represent the model using a statistical shape model; and
performing standard lossless compression.

101. The method of any one of claims 92 to 100 comprising performing planning operations to define the planning data.

102. The method of any one of claims 92 to 101 comprising receiving the planning data from a planning system configured to define the planning data.
